(19) United States
(12) Patent Application Publication
(10) Pub. No.: US 2010/0137381 A1
(43) Pub. Date: Jun. 3, 2010

(54) ACETAMIDE DERIVATIVES AS POTASSIUM CHANNEL MODULATORS

(75) Inventors: Antonio Nardi, Ballerup (DK); Morten Grunnet, Kobenhavn (DK); Palle Christophersen, Ballerup (DK); Joachim Demnitz, Kobenhavn (DK); David Spencer Jones, Smorum (DK); Elsebet Østergaard Nielsen, Kobenhavn (DK); Dorte Strøbæk, Farum (DK); Lars Siim Madsen, Soro (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/598,541

(22) PCT Filed: Apr. 29, 2008
(86) PCT No.: PCT/EP08/55215

§ 371 Date: Jan. 20, 2010

Related U.S. Application Data
(60) Provisional application No. 60/916,101, filed on May 4, 2007.

(30) Foreign Application Priority Data
May 3, 2007 (DK) PA 2007 00668

(51) Int. Cl. A61K 31/4245; C07D 257/04; A61K 31/41; C07D 271/06; A61P 3/10; A61P 9/10; A61P 9/00; A61P 9/06; A61P 35/00; A61P 25/06; A61P 25/16; A61P 25/28; A61P 25/22; A61P 25/24

(52) U.S. Cl. 514/364; 548/253; 514/381; 548/131

(57) ABSTRACT

This invention relates to novel acetamide derivatives that are found to be potent modulators of ion channels, in particular potassium channels and chloride channels, and, as such, are valuable candidates for the treatment of diseases or disorders as diverse as those which are responsive to the modulation of potassium channels.

### TECHNICAL FIELD

This invention relates to novel acetamide derivatives that are found to be potent modulators of ion channels, in particular potassium channels and chloride channels, and, as such, are valuable candidates for the treatment of diseases or disorders as diverse as those which are responsive to the modulation of potassium channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

Chloride channels serve a wide variety of specific cellular functions and contribute to the normal function of i.a. skeletal and smooth muscle cells. Chloride channels are probably found in every cell, from bacteria to mammals. Their physiological tasks range from cell volume regulation to stabilization of the membrane potential, transepithelial or transcellular transport and acidification of intracellular organelles.

WO 2007/044724 describes certain N-tetrazolylphenyl carboxamide derivatives useful as PIM-1 and PIM-3 protein kinase inhibitors. However, the acetamide derivatives of the present invention are not described, and their use as potassium channel modulators certainly not suggested.

### SUMMARY OF THE INVENTION

Is an object of the invention to provide novel acetamide derivatives useful as ion channel modulators. The acetamide derivatives of the invention may be characterised by Formula I

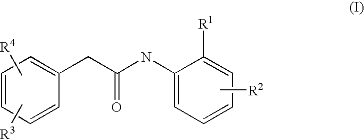

a stereoisomer or a mixture of its stereoisomers, or a pharmaceutically-acceptable addition salt thereof, wherein

R¹ represents a tetrazolyl, an N-hydroxy-carbamimidoyl or a 5-oxo-4,5-dihydro-[1,2,4]oxadiazol-3-yl group;

R² represents halo, trifluoromethyl or phenyl, which phenyl may optionally be substituted one or two times with halo, trifluoromethyl, trifluoromethoxy and/or N,N-dialkylsulfamoyl; and

R³ and R⁴, independently of each other, represent hydrogen, halo or trifluoromethyl hydroxy, alkylsulfonyl or SO₂NR′R″, wherein R′ and R″ represents hydrogen or alkyl, or R′ and R″, together with the N-atom to which they are attached, form a heterocyclic ring selected from piperidine, piperazine and morpholine.

In another aspect the invention provides pharmaceutical compositions comprising a therapeutically effective amount of an acetamide derivative of the invention.

In a third aspect the invention relates to the use of the acetamide derivatives of the invention for the manufacture of pharmaceutical compositions.

In a further aspect the invention provides a method of treatment, prevention or alleviation of a disease or a disorder or a condition of a living animal body, including a human, which disorder, disease or condition is responsive to modulation of ion channels, which method comprises the step of administering to such a living animal body in need thereof, a therapeutically effective amount of the acetamide derivative of the invention.

Other objects of the invention will be apparent to the person skilled in the art from the following detailed description and examples.

### DETAILED DISCLOSURE OF THE INVENTION

In its first aspect the invention provides novel acetamide derivatives of Formula I

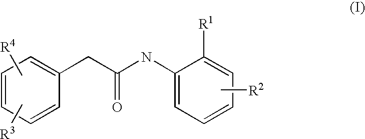

a stereoisomer or a mixture of its stereoisomers, or a pharmaceutically-acceptable addition salt thereof, wherein

R¹ represents a tetrazolyl, an N-hydroxy-carbamimidoyl or a 5-oxo-4,5-dihydro-[1,2,4]oxadiazol-3-yl group;

R² represents halo, trifluoromethyl or phenyl, which phenyl may optionally be substituted one or two times with halo, trifluoromethyl, trifluoromethoxy and/or N,N-dialkylsulfamoyl; and

R³ and R⁴, independently of each other, represent hydrogen, halo or trifluoromethyl hydroxy, alkylsulfonyl or SO₂NR′R″, wherein R′ and R″ represents hydrogen or alkyl, or R′ and R″, together with the N-atom to which they are attached, form a heterocyclic ring selected from piperidine, piperazine and morpholine;

provided, however, if R¹ represents tetrazolyl, R² represents chloro, and one of R³ and R⁴ represents hydrogen or bromo; then the other of R³ and R⁴ does not represent hydrogen.

In a more preferred embodiment the acetamide derivative of the invention is a compound of Formula Ia

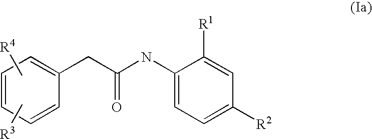

a stereoisomer or a mixture of its stereoisomers, or a pharmaceutically-acceptable addition salt thereof, wherein R¹, R², R³ and R⁴ are as defined above.

In another more preferred embodiment the acetamide derivative of the invention is a compound of Formula Ib

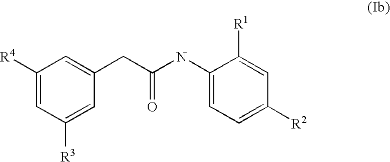

a stereoisomer or a mixture of its stereoisomers, or a pharmaceutically-acceptable addition salt thereof, wherein R¹, R², R³ and R⁴ are as defined above.

In an even more preferred embodiment the acetamide derivative of the invention is a compound of Formula Ib, wherein

R¹ and R² are as defined above; and

R³ and R⁴, independently of each other, represent halo, in particular fluoro or chloro, or trifluoromethyl.

In a third more preferred embodiment the acetamide derivative of the invention is a compound of Formula Ic

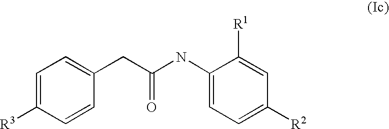

a stereoisomer or a mixture of its stereoisomers, or a pharmaceutically-acceptable addition salt thereof, wherein

R¹ and R² are as defined above; and

R³ represents halo, in particular chloro, or trifluoromethyl.

In an even more preferred embodiment the acetamide derivative of the invention is a compound of Formula Ic, wherein R³ represents halo, and in particular chloro.

In another preferred embodiment the acetamide derivative of the invention is a compound of Formula I, Ia, Ab or Ic, or a pharmaceutically-acceptable addition salt thereof, wherein R¹ represents a tetrazolyl, an N-hydroxy-carbamimidoyl or a 5-oxo-4,5-dihydro-[1,2,4]oxadiazol-3-yl group.

In a more preferred embodiment R¹ represents a tetrazolyl group, in particular a 1H-tetrazol-5-yl group.

In another more preferred embodiment R¹ represents an N-hydroxy-carbamimidoyl group.

In a third more preferred embodiment R¹ represents a 5-oxo-4,5-dihydro-[1,2,4]oxadiazol-3-yl group.

In a third preferred embodiment the acetamide derivative of the invention is a compound of Formula I, Ia, Ab or Ic, or a pharmaceutically-acceptable addition salt thereof, wherein R² represents halo, trifluoromethyl or phenyl, which phenyl may optionally be substituted one or two times with halo, trifluoromethyl, trifluoromethoxy and/or N,N-dialkylsulfamoyl.

In a more preferred embodiment R² represents halo, in particular chloro or bromo, or phenyl, which phenyl may optionally be substituted one or two times with halo, in particular fluoro, trifluoromethyl, trifluoromethoxy and/or N,N-dialkylsulfamoyl.

In an even more preferred embodiment R² represents halo or trifluoromethyl.

In another more preferred embodiment R² represents halo, and in particular chloro or bromo.

In a third more preferred embodiment R² represents phenyl, which phenyl may optionally be substituted with halo, in particular fluoro, trifluoromethyl, trifluoromethoxy or N,N-dialkylsulfamoyl.

In an even more preferred embodiment R² represents phenyl.

In a fourth more preferred embodiment R² represents a phenyl group substituted with halo, in particular fluoro, trifluoromethyl, trifluoromethoxy or N,N-dialkylsulfamoyl.

In an even more preferred embodiment R² represents a phenyl group substituted with halo, in particular fluoro.

In a fifth more preferred embodiment R² represents a phenyl group substituted with trifluoromethyl.

In a sixth more preferred embodiment R² represents a phenyl group substituted with trifluoromethoxy.

In a seventh more preferred embodiment R² represents a phenyl group substituted with N,N-dialkylsulfamoyl, in particular N,N-dimethylsulfamoyl.

In a fourth preferred embodiment the acetamide derivative of the invention is a compound of Formula I, Ia, Ab or Ic, or a pharmaceutically-acceptable addition salt thereof, wherein R³ and R⁴, independently of each other, represent hydrogen, halo, trifluoromethyl, hydroxy, alkylsulfonyl or SO₂NR′R″, wherein R′ and R″ represents hydrogen or alkyl, or R′ and R″, together with the N-atom to which they are attached, form a heterocyclic ring selected from piperidine, piperazine and morpholine.

In a more preferred embodiment R³ and R⁴, independently of each other, represent hydrogen, halo, trifluoromethyl, hydroxy, alkylsulfonyl or SO₂NR′R″, wherein R′ and R″ together with the N-atom to which they are attached, form a piperidine ring.

R³ and R⁴, independently of each other, represent hydrogen, halo, in particular fluoro or chloro, or trifluoromethyl.

In a more preferred embodiment R³ and R⁴ both represent halo, in particular fluoro or chloro, or trifluoromethyl.

In another more preferred embodiment R³ and R⁴ both represent halo, in particular fluoro or chloro.

In a third more preferred embodiment R³ and R⁴ both represent trifluoromethyl.

In a most preferred embodiment the acetamide derivative of the invention is
2-(3,5-Difluoro-phenyl)-N-[3-(5-oxo-4,5-dihydro-[1,2,4]oxadiazol-3-yl)-4′-trifluoromethyl-biphenyl-4-yl]-acetamide;
N-[4-Bromo-2-(1H-tetrazol-5-yl)-phenyl]-2-(3,5-difluoro-phenyl)-acetamide;
2-(3,5-Bis-trifluoromethyl-phenyl)-N-[4-bromo-2-(1H-tetrazol-5-yl)-phenyl]-acetamide;
2-(4-Chloro-phenyl)-N-[4′-chloro-3-(1H-tetrazol-5-yl)-biphenyl-4-yl]-acetamide;
2-(4-Chloro-phenyl)-N-[4′-fluoro-3-(1H-tetrazol-5-yl)-biphenyl-4-yl]-acetamide;
2-(4-Chloro-phenyl)-N-[4′-dimethylsulfamoyl-3-(1H-tetrazol-5-yl)-biphenyl-4-yl]-acetamide;
2-(4-Chloro-phenyl)-N-[3-(5-oxo-4,5-dihydro-[1,2,4]oxadiazol-3-yl)-4′-trifluoromethyl-biphenyl-4-yl]-acetamide;
2-(4-Chloro-phenyl)-N-[3-(1H-tetrazol-5-yl)-4′-trifluoromethoxy-biphenyl-4-yl]-acetamide;
N-[5-Chloro-2-(5-oxo-4,5-dihydro-[1,2,4]oxadiazol-3-yl)-phenyl]-2-[4-(piperidine-1-sulfonyl)-phenyl]-acetamide; or
N-[5-Chloro-2-(5-oxo-4,5-dihydro-[1,2,4]oxadiazol-3-yl)-phenyl]-2-(3-methanesulfonyl-phenyl)-acetamide;

or a pharmaceutically-acceptable addition salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

### Pharmaceutically Acceptable Salts

The acetamide derivatives of the invention may be provided in any form suitable for the intended administration. Suitable forms include pharmaceutically (i.e. physiologically) acceptable salts, and pre- or prodrug forms of the acetamide derivative of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of an acetamide derivative of the invention include, without limitation, the sodium, the potassium, the calcium, the magnesium, the lithium, and the ammonium salt, and the like, of an acetamide derivative of the invention containing an anionic group. Such cationic salts may be formed by procedures well known and described in the art.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such isomers and any mixtures thereof including racemic mixtures.

Racemic forms can be resolved into the optical antipodes by known methods and techniques. One way of resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J, Collet A, & Wilen S in “Enantiomers. Racemates, and Resolutions”, John Wiley and Sons, New York (1981). Optical active compounds can also be prepared from optically active starting materials or intermediates.

### Methods of Preparation

The compounds according to the invention may be prepared by conventional methods for chemical synthesis, e.g. those described in the working examples.

### Biological Activity

The acetamide derivatives of the invention have been found to possess ion channel modulating activity, and in particular potassium channel activating activity and chloride channel blocking activity, as measured by standard electrophysiological methods. Due to their activity at the potassium and chloride channels, the acetamide derivatives of the invention are considered useful for the treatment of a wide range of diseases and conditions.

In a special embodiment, the acetamide derivatives of the invention are considered useful for the treatment, prevention or alleviation of a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjogren's syndrome, xerostomia, arrhythmia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

In a more preferred embodiment, the acetamide derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the acetamide derivatives of the invention are considered useful for the treatment, prevention or alleviation of psychosis, schizophrenia, bipolar disorder, depression, epilepsy, Parkinson's disease or pain.

In a third more preferred embodiment, the acetamide derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the acetamide derivatives of the invention are considered useful for the treatment, prevention or alleviation of cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy, cardiomyopathy or a genetic disease.

In a fifth more preferred embodiment, the acetamide derivatives of the invention are considered useful for the treatment, prevention or alleviation of cardiac ischemia, ischemic heart disease, hypertrophic heart, cardiomyopathy or failing heart.

In a sixth more preferred embodiment, the compounds of the invention are considered useful for the treatment, prevention or alleviation of a cardiovascular disease. In a more preferred embodiment the cardiovascular disease is atherosclerosis, ischemia/reperfusion, hypertension, restenosis, arterial inflammation, myocardial ischaemia or ischaemic heart disease.

In a seventh more preferred embodiment, the acetamide derivatives of the invention are considered useful for the treatment, prevention or alleviation of cardiac arrhythmia, atrial fibrillation and/or ventricular tachyarrhythmia.

In an eighth more preferred embodiment, the compounds of the invention are considered useful for obtaining preconditioning of the heart. Preconditioning, which includes ischemic preconditioning and myocardial preconditioning, describes short periods of ischemic events before initiation of a long lasting ischemia. The compounds of the invention are believed having an effect similar to preconditioning obtained by such ischemic events. Preconditioning protects against later tissue damage resulting from the long lasting ischemic events.

In a ninth more preferred embodiment, the acetamide derivatives of the invention are considered useful for the treatment, prevention or alleviation of schizophrenia, depression or Parkinson's disease.

In a tenth more preferred embodiment, the compounds of the invention are considered useful for the treatment, prevention or alleviation of an obstructive or inflammatory airway disease. In a more preferred embodiment the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis.

In its most preferred embodiment the obstructive airway disease is chronic obstructive pulmonary disease (COPD).

In an eleventh more preferred embodiment, the acetamide derivatives of the invention are considered useful for the treatment, prevention or alleviation of a sexual dysfunction, incl. male sexual dysfunction and female sexual dysfunction, and incl. male erectile dysfunction.

In an even more preferred embodiment the acetamide derivative of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the acetamide derivative of the invention is used in a combination therapy together with sildenafil, tadalafil, vardenafil or calcium dobesilate.

In a twelfth more preferred embodiment, the acetamide derivatives of the invention are considered useful for the treatment, prevention or alleviation of ophthalmic angiogenesis related diseases, disorders or conditions, such as exudative macular degeneration, age-related macular degeneration (AMD), retinopathy, diabetic retinopathy, proliferative diabetic retinopathy, diabetic macular edema (DME), ischemic retinopathy (e.g. retinal vain or artery occlusion), retinopathy of prematurity, neovascular ocular hypertension, glaucoma and corneal neovascularization. In the context of this invention, “age-related macular degeneration” (AMD) includes dry AMD (non-exudative AMD) and wet AMD (exudative AMD). In a special embodiment, the invention relates to treatment, prevention or alleviation of wet AMD.

The acetamide derivatives of the invention are considered particular useful for the treatment of a disease, disorder or condition that is responsive to reduction of intraocular pressure, such as ocular hypertension, open-angle glaucoma, chronic open-angle glaucoma, angle-closure glaucoma and ciliary injection caused by angle-closure glaucoma.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred acetamide derivatives of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of an acetamide derivative of the invention.

While an acetamide derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the acetamide derivative of the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Pharmaceutical Kits of Parts

According to the invention there is also provided a kit of parts comprising at least two separate unit dosage forms (A) and (B):

(A) an acetamide derivative of the invention; and

(B1) a phosphodiesterase inhibitor; or

(B2) an agent that potentiates endothelium-derived hyperpolarizing factor-mediated responses; and optionally

(C) instructions for the simultaneous, sequential or separate administration of the acetamide derivative of A, and the phosphodiesterase inhibitor of B1, or an agent that potentiates endothelium-derived hyperpolarizing factor-mediated responses of B2, to a patient in need thereof.

In a more preferred embodiment the phosphodiesterase inhibitor for use according to the invention (B1) is a phosphodiesterase 5 (PDE5) inhibitor, and in an even more preferred embodiment the phosphodiesterase inhibitor for use according to the invention is sildenafil, tadalafil or vardenafil.

In another more preferred embodiment the agent that potentiates endothelium-derived hyperpolarizing factor-mediated responses for use according to the invention (B2) is calcium dobesilate.

The acetamide derivative of the invention and the phosphodiesterase inhibitor or the agent that potentiates endothelium-derived hyperpolarizing factor-mediated responses for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the acetamide derivative of the invention and the phosphodiesterase inhibitor or the agent that potentiates endothelium-derived hyperpolarizing factor-mediated responses for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the positive allosteric nicotine receptor modulator and the cognitive enhancer are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Therapy

In another aspect the invention provides a method of treatment, prevention or alleviation of a disease, disorder or condition of a living animal body, including a human, which disorder, disease or condition is responsive to modulation of an ion channel, and in particular a potassium channel or a chloride channel, which method comprises the step of administering to such a living animal body in need thereof, a therapeutically effective amount a compound capable of activating the potassium channel, or a pharmaceutically-acceptable addition salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWING

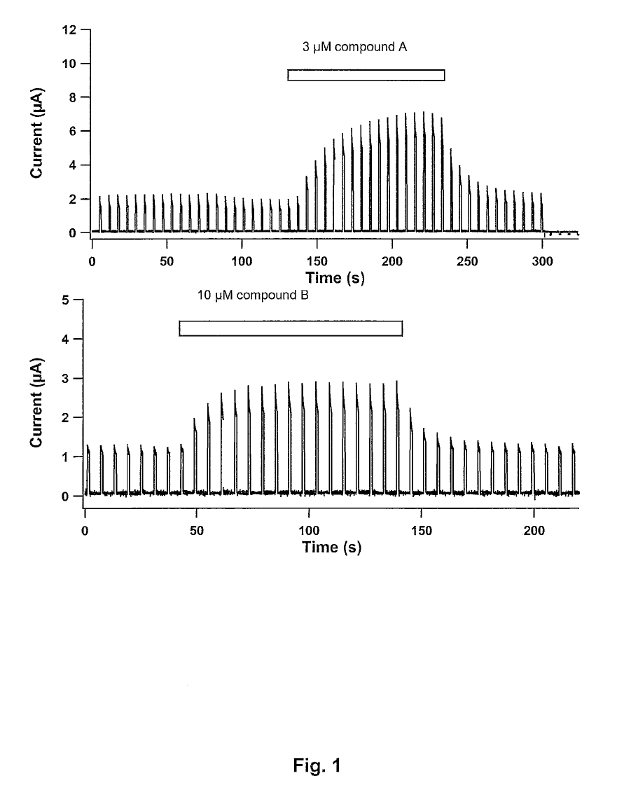

The present invention is further illustrated by reference to the accompanying drawing, in which FIG. 1 shows the BK channel opening activity [current (μA) vs. time (s)] of two acetamide derivatives representative of the invention, i.e. 2-(3,5-difluoro-phenyl)-N-[3-(5-oxo-4,5-dihydro-[1,2,4]oxadiazol-3-yl)-4′-trifluoromethyl-biphenyl-4-yl]-acetamide (Compound 7), and 2-(3,5-bis-trifluoromethyl-phenyl)-N-[4-bromo-2-(1H-tetrazol-5-yl)-phenyl]-acetamide (Compound 2) herein designated Compound A and Compound B, respectively, determined by a standard electrophysiological method using BK channels heterologously expressed in Xenopus laevis oocytes.

### EXAMPLES

The invention is further illustrated with reference to the following examples, which are not intended to be in any way limiting to the scope of the invention as claimed.

### Example 1

### Preparatory Example

### General Experimental Procedure

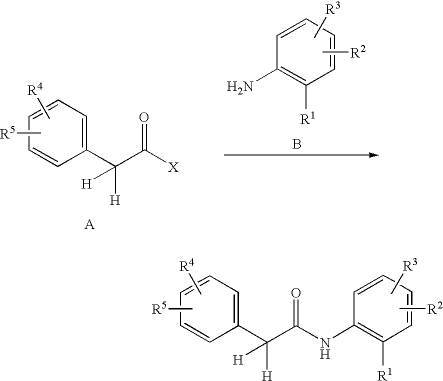

The synthetic pathway of the acetamide compounds of the invention involves amide formation by coupling a suitably-substituted phenylacetic acid (A, X═OH) with an ortho-substituted aniline (B) in presence of EDC (1-Ethyl-3-[3-dimethylaminopropyl]carbodiimide hydrochloride) or coupling a suitably-substituted phenylacetylchloride (A, X═Cl), commercially-available or prepared from the corresponding commercial phenylacetic acids by treatment with thionyl chloride, with an ortho-substituted aniline (B). When the aniline (B) was not commercially-available it was synthesised either as described in e.g. WO 98/47879 and in Valgeirsson et al. in Journal of Medicinal Chemistry 2004 47 (27) 6948-6957 or by the palladium catalyzed Suzuki cross-coupling reaction between a halogenated aniline and a suitably-substituted arylboronic acid. In case the starting halogenated aniline is substituted by a ciano group in the ortho position, the Suzuki cross-coupling reaction is followed by the conversion of the cyano moiety to the correspondent tetrazolyl or 5-oxo-4,5-dihydro-[1,2,4]oxadiazol-3-yl derivative, as described in Valgeirsson et al. in Journal of Medicinal Chemistry 2004 47 (27) 6948-6957. As an example of the synthetic experimental procedure for a non-commercial-aniline derivative B, the synthesis of the intermediate D is reported.

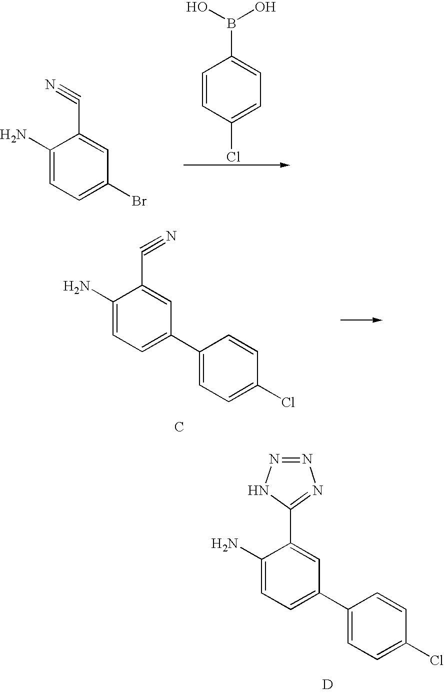

### 4-Amino-4′-chloro-biphenyl-3-carbonitrile (C)

To a mixture of the commercial 2-amino-5-bromo-benzonitrile (5.5 g, 1 eq), 4-chlorobenzeneboronic acid (4.8 g, 1.1 eq), potassium carbonate (12.7 g, 3.3 eq), dimethoxy ethane (80 ml) and water (40 ml), bistriphenylphosphine palladium (II) chloride (0.2 g) is added. The resulting mixture is refluxed for 24 hours and then evaporated to dryness. The residue is purified by flash chromatography using dichloromethane as eluent (5.32 g, 83% yield).

### 4′-Chloro-3-(1H-tetrazol-5-yl)-biphenyl-4-ylamine (D)

A mixture of 4-amino-4′-chloro-biphenyl-3-carbonitrile (5.3 g, 1 eq), sodium azide (2.3 g, 1.5 eq) and trethylamine hydrochloride (4.9 g, 1.5 eq) is suspended in 40 ml of toluene and heated (60° C.) overnight. To the reaction mixture, cooled to room temperature, water and 4M HCl are added, to afford the title compound as a white solid. This is collected by filtration (4.83 g, 77% yield) and used for the next step without further purification.

### N-[4-Bromo-2-(1H-tetrazol-5-A-phenyl]-2-(3,5-difluoro-phenyl)-acetamide (1)

To a stirred solution of 4-bromo-2-(1H-tetrazol-5-yl)-phenyl amine (0.315 g) in pyridine (6 ml), the commercial 2-(3,5-difluorophenyl)ethanoyl chloride (0.25 g, 1 eq) is added portion-wise and stirring is continued at room temperature for 4 hours.

The resulting reaction mixture is evaporated to dryness and the solid residue is washed with HCl 1N and water, and then purified by crystallization from acetonitrile (0.35 g, 68% yield). LC-ESI-HRMS of [M−H]− shows 391.9947 Da. Calc. 391.995854 Da, dev. −2.9 ppm.

### 2-(3,5-Bis-trifluoromethyl-phenyl)-N-[4-bromo-2-(1H-tetrazol-5-yl)-phenyl]-acetamide (2)

To a stirred solution of 4-bromo-2-(1H-tetrazol-5-yl)-phenyl amine (0.44 g) in pyridine (12 ml), the commercial 3,5-bis(trifluoromethyl)phenylacetyl chloride (0.53 g, 1 eq) is added portion-wise and stirring at room temperature is continued overnight.

The resulting reaction mixture is evaporated to dryness and the solid residue is washed first with HCl 1N and water, and then purified by prep-LCMS (0.42 g, 46% yield). LC-ESI-HRMS of [M−H]− shows 491.9892 Da. Calc. 491.989466 Da, dev. −0.5 ppm.

### 2-(4-Chloro-phenyl)-N-[4′-chloro-3-(1H-tetrazol-5-yl)-biphenyl-4-yl]acetamide (3)

To a stirred solution of 4′-chloro-3-(1H-tetrazol-5-yl)-biphenyl-4-ylamine (0.40 g) in pyridine (12 ml), a solution of the commercial 4-chlorophenylacetylchloride (0.28 g, 1 eq) is added portion-wise and stirring is continued overnight at 50° C. The resulting reaction mixture is evaporated to dryness and the solid residue is washed first with HCl 1N and water, and then purified by crystallization from ethanol/water (0.45 g, 72% yield). LC-ESI-HRMS of [M−H]− shows 422.0561 Da. Calc. 422.057541 Da, dev. −3.4 ppm.

### 2-(4-Chloro-phenyl)-N-[4′-fluoro-3-(1H-tetrazol-5-yl)-biphenyl-4-yl]-acetamide (4)

To a stirred solution of 4′-Fluoro-3-(1H-tetrazol-5-yl)-biphenyl-4-ylamine (0.337 g) in pyridine (12 ml), a solution of commercial 4-chlorophenylacetylchloride (0.25 g, 1 eq) is added portion-wise and stirring is continued overnight at 50° C. The resulting reaction mixture is evaporated to dryness and the solid residue is washed first with HCl 1N and water, and then purified by crystallization from ethanol (0.46 g, 85% yield). LC-ESI-HRMS of [M−H]− shows 406.0852 Da. Calc. 406.087091 Da, dev. −4.7 ppm

### 2-(4-Chloro-phenyl)-N-[4′-dimethylsulfamoyl-3-(1H-tetrazol-5-yl)-biphenyl-4-yl]-acetamide (5)

To a stirred solution of 4′-Amino-3′-(1H-tetrazol-5-yl)-biphenyl-4-sulfonic acid dimethylamide (1.09 g) in pyridine (25 ml), a solution of the commercial 4-chlorophenylacetylchloride (0.6 g, 1 eq) is added portion-wise and stirring is continued overnight at 50° C. The resulting reaction mixture is evaporated to dryness and the solid residue is washed first with HCl 1N and water, and then purified by crystallization from ethanol/water (0.72 g, 48% yield). LC-ESI-HRMS of [M−H]− shows 495.1009 Da. Calc. 495.100613 Da, dev. 0.6 ppm.

### 2-(4-Chloro-phenyl)-N-[3-(1H-tetrazol-5-yl)-4′-trifluoromethoxy-biphenyl-4-yl]-acetamide (6)

To a stirred solution of 3-(1H-tetrazol-5-yl)-4′-trifluoromethoxy-biphenyl-4-ylamine (0.34 g) in pyridine (10 ml), a solution of the commercial 4-chlorophenylacetylchloride (0.2 g, 1 eq) is added portion-wise and stirring is continued overnight at 50° C. The resulting reaction mixture is evaporated to dryness and the solid residue is washed first with HCl 1N and water, and then purified by crystallization from ethanol (0.32 g, 62% yield). LC-ESI-HRMS of [M−H]− shows 472.0779 Da. Calc. 472.078812 Da, dev. −1.9 ppm.

### 2-(3,5-Difluoro-phenyl)-N-[3-(5-oxo-4,5-dihydro-[1,2,4]oxadiazol-3-yl)-4′-trifluoromethyl-biphenyl-4-yl]acetamide (7)

To a stirred solution of 3-(4-Amino-4′-trifluoromethyl-biphenyl-3-yl)-4H-[1,2,4]oxadiazol-5-one (0.42 g) in pyridine (10 ml), a solution of commercial 2-(3,5-difluorophenyl)ethanoyl chloride (0.25 g, 1 eq) is added portion-wise and stirring is continued overnight at 50° C. The resulting reaction mixture is evaporated to dryness and the solid residue is washed first with HCl 1N and water, and then purified by flash chromatography using ethyl acetate (20%) and petroleum ether (80%) as eluent, to afford the title compound as yellowish powder (0.16 g, 19% yield). LC-ESI-HRMS of [M−H]− shows 474.0875 Da. Calc. 474.087707 Da, dev. −0.4 ppm.

### 2-(4-Chloro-phenyl)-N-[3-(5-oxo-4,5-dihydro-[1,2,4]oxadiazol-3-yl)-4′-trifluoromethyl-biphenyl-4-yl]acetamide (8)

To a stirred solution of 3-(4-Amino-4′-trifluoromethyl-biphenyl-3-yl)-4H-[1,2,4]oxadiazol-5-one (0.09 g) in pyridine (3 ml), a solution of the commercial 4-chlorophenylacetylchloride (0.053 g, 1 eq) is added portion-wise and stirring is continued overnight at 50° C. The resulting reaction mixture is evaporated to dryness and the solid residue is washed first with HCl 1N and water, and then purified by crystallisation fro ethyl acetate/petroleum ether (0.023 g, 17% yield). LC-ESI-HRMS of [M−H]− shows 472.068 Da. Calc. 472.067579 Da, dev. 0.9 ppm.

### N-[5-Chloro-2-(5-oxo-4,5-dihydro-[1,2,4]oxadiazol-3-yl)-phenyl]-2-[4-(piperidine-1-sulfonyl)-phenyl]-acetamide (9)

To a suspension of commercial (4-(piperidine-1-sulfonyl)-phenyl)-acetic acid (0.25 g, 1 eq) in dichloromethane (15 ml), EDC.HCl (0.338 g, 2 eq) and DMAP (0.323 g, 3 eq) are added. The resulting brown solution is stirred for 10 min and 3-(2-amino-4-chloro-phenyl)-4H-[1,2,4]oxadiazol-5-one (0.186 g, 1 eq) prepared as described by Valgeirsson et al. in Journal of Medicinal Chemistry 2004 47 (27) 6948-6957 is then added. The reaction mixture is stirred at room temperature overnight, diluted with dichloromethane (20 ml), washed with 1.5 N HCl (2×25 ml) and water (25 ml) and finally dried over MgSO₄ and evaporated to dryness, to give a yellowish solid. This crude material is purified by preparative HPLC (0.090 g, yield 21%), to afford the title compound as white powder. LC-ESI-HRMS of [M+H]+ shows 477.1008 Da. Calc. 477.099945 Da, dev. 1.8 ppm.

### N-[5-Chloro-2-(5-oxo-4,5-dihydro-[1,2,4]oxadiazol-3-yl)-phenyl]-2-(3-methanesulfonyl-phenyl)-acetamide (10)

To a suspension of commercial 3-(methylsulfonyl)phenylacetic acid (0.25 g, 1 eq) in dichloromethane (15 ml), EDC.HCl (0.447 g, 2 eq) and DMAP (0.428 g, 3 eq) are added. The resulting brown solution is stirred for 10 min and 3-(2-amino-4-chloro-phenyl)-4H-[1,2,4]oxadiazol-5-one (0.247 g, 1 eq) prepared as described by Valgeirsson et al. in Journal of Medicinal Chemistry 2004 47 (27) 6948-6957 is then added. The reaction mixture is stirred at room temperature overnight, diluted with dichloromethane (20 ml), washed with 1.5 N HCl (2×25 ml) and water (25 ml) and finally dried over MgSO₄ and evaporated to dryness, to give a yellowish solid. This crude material is purified by preparative HPLC (0.150 g, yield 31%), to afford the title compound as white powder. LC-ESI-HRMS of [M+H]+ shows 408.0413 Da. Calc. 408.042096 Da, dev. −2 ppm.

### Example 2

### BK Channel Activation

In this example the BK channel opening activity of two acetamide derivatives of to the invention, i.e. 2-(3,5-difluoro-phenyl)-N-[3-(5-oxo-4,5-dihydro-[1,2,4]oxadiazol-3-yl)-4′-trifluoromethyl-biphenyl-4-yl]-acetamide (Compound 7), and 2-(3,5-bis-trifluoromethyl-phenyl)-N-[4-bromo-2-(1H-tetrazol-5-yl)-phenyl]-acetamide (Compound 2) herein designated Compound A and Compound B, respectively, is determined using BK channels heterologously expressed in Xenopus laevis oocytes.

The electrical current through the BK channel is measured by conventional two-electrode voltage clamp. BK current is activated by repeated step protocols. In brief, this protocol goes from a resting membrane potential of −40 mV lasting for 5 s to a depolarised step to +20 mV lasting for 1 s. The protocol was repeated continuously.

Having reached a stable current level, Compound A (3 μM) and Compound B (10 μM), respectively, was added. A marked increase in the current activated by depolarisation could be observed. The BK current activity returned to baseline after approximately 30-80 seconds of wash. In summary BK current was increased by 367±92% in the presence of 3 μM of Compound A (n=3, SD value), and 327±88% in the presence of 10 μM of Compound B.

The results are presented in FIG. 1.

### Example 3

### In Vitro Human Erythrocyte Chloride Conductance

In this example the chloride channel blocking activity of an acetamide derivative representative of the invention, i.e. Compound 3 (2-(4-Chloro-phenyl)-N-[4′-chloro-3-(1H-tetrazol-5-yl)-biphenyl-4-yl]-acetamide), has been determined.

All dose-response experiments were therefore performed by concomitant measurements of conductive netfluxes of Cl⁻ (J_{Cl}) and membrane potentials (Vₘ) in suspensions of erythrocytes as described by Bennekou et al. (Bennekou P and Christophersen P: Flux ratio of Valinomycin—Mediated K⁺Fluxes across the Human Red Cell Membrane in the presence of the Protronophore CCCP; J. Membrane Biol. 1986 93 221-227).

The membrane Cl-conductances (G_{Cl}) were calculated according to Hodgkin et al. (Hodgkin A L and Huxley A F: The components of membrane conductance in the giant axon of Loligo; J. Physiol. Lond. 1952 116 449-472) using the following equation:

$${G}_{Cl} = \frac{F * {J}_{Cl}}{( {V}_{m} - {E}_{Cl} )}$$

where F is the Faraday constant and E_{Cl} is the Nernst potential for the Cl-ion.

The K_{D}-value for Compound 3 was calculated as 0.090 μM.

1-9. (canceled)

10. An acetamide derivative of Formula I 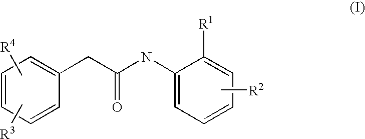
a stereoisomer or a mixture of its stereoisomers, or a pharmaceutically-acceptable addition salt thereof, wherein
R¹ represents a tetrazolyl, an N-hydroxy-carbamimidoyl or a 5-oxo-4,5-dihydro-[1,2,4]oxadiazol-3-yl group;
R² represents halo, trifluoromethyl or phenyl, which phenyl may optionally be substituted one or two times with halo, trifluoromethyl, trifluoromethoxy and/or N,N-dialkylsulfamoyl; and
R³ and R⁴, independently of each other, represent hydrogen, halo or trifluoromethyl hydroxy, alkylsulfonyl or SO₂NR′R″, wherein R′ and R″ represents hydrogen or alkyl, or R′ and R″, together with the N-atom to which they are attached, form a heterocyclic ring selected from piperidine, piperazine and morpholine.

11. The acetamide derivative of claim 10, a stereoisomer or a mixture of its stereoisomers, or a pharmaceutically-acceptable addition salt thereof, wherein R¹ represents a tetrazolyl, an N-hydroxy-carbamimidoyl or a 5-oxo-4,5-dihydro-[1,2,4]oxadiazol-3-yl group.

12. The acetamide derivative of claim 10, a stereoisomer or a mixture of its stereoisomers, or a pharmaceutically-acceptable addition salt thereof, wherein R² represents halo, trifluoromethyl or phenyl, which phenyl may optionally be substituted one or two times with halo, trifluoromethyl, trifluoromethoxy and/or N,N-dialkylsulfamoyl.

13. The acetamide derivative of claim 10, a stereoisomer or a mixture of its stereoisomers, or a pharmaceutically-acceptable addition salt thereof, wherein R³ and R⁴, independently of each other, represent hydrogen, halo, trifluoromethyl, hydroxy, alkylsulfonyl or SO₂NR′R″, wherein R′ and R″ represents hydrogen or alkyl, or R′ and R″, together with the N-atom to which they are attached, form a heterocyclic ring selected from piperidine, piperazine and morpholine.

14. The acetamide derivative of claim 10, which is
2-(3,5-Difluoro-phenyl)-N-[3-(5-oxo-4,5-dihydro-[1,2,4]oxadiazol-3-yl)-4′-trifluoromethyl-biphenyl-4-yl]-acetamide;
N-[4-Bromo-2-(1H-tetrazol-5-yl)-phenyl]-2-(3,5-difluoro-phenyl)-acetamide;
2-(3,5-Bis-trifluoromethyl-phenyl)-N-[4-bromo-2-(1H-tetrazol-5-yl)-phenyl]-acetamide;
2-(4-Chloro-phenyl)-N-[4′-chloro-3-(1H-tetrazol-5-yl)-biphenyl-4-yl]-acetamide;
2-(4-Chloro-phenyl)-N-[4′-fluoro-3-(1H-tetrazol-5-yl)-biphenyl-4-yl]-acetamide;
2-(4-Chloro-phenyl)-N-[4′-dimethylsulfamoyl-3-(1H-tetrazol-5-yl)-biphenyl-4-yl]-acetamide;
2-(4-Chloro-phenyl)-N-[3-(5-oxo-4,5-dihydro-[1,2,4]oxadiazol-3-yl)-4′-trifluoromethyl-biphenyl-4-yl]-acetamide;
2-(4-Chloro-phenyl)-N-[3-(1H-tetrazol-5-yl)-4′-trifluoromethoxy-biphenyl-4-yl]-acetamide;
N-[5-Chloro-2-(5-oxo-4,5-dihydro-[1,2,4]oxadiazol-3-yl)-phenyl]-2-[4-(piperidine-1-sulfonyl)-phenyl]-acetamide; or
N-[5-Chloro-2-(5-oxo-4,5-dihydro-[1,2,4]oxadiazol-3-yl)-phenyl]-2-(3-methanesulfonyl-phenyl)-acetamide;
a stereoisomer or a mixture of its stereoisomers, or a pharmaceutically-acceptable addition salt thereof.

15. A pharmaceutical composition comprising a therapeutically effective amount of the acetamide derivative of claim 10, a stereoisomer or a mixture of its stereoisomers, or a pharmaceutically-acceptable addition salt thereof, together with one or more adjuvants, excipients, carriers and/or diluents.

16. A method of treatment, prevention or alleviation of a disease or a disorder or a condition of a living animal body, including a human, which disorder, disease or condition is responsive to modulation of ion channels, which method comprises the step of administering to such a living animal body in need thereof, a therapeutically effective amount of the acetamide derivative according to claim 10, a stereoisomer or a mixture of its stereoisomers, or a pharmaceutically-acceptable addition salt thereof.

17. The method according to claim 16, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjogren's syndrome, xerostomia, arrhythmia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.
